(19) United States
(12) Patent Application Publication
(10) Pub. No.: US 2018/0289467 A1
(43) Pub. Date: Oct. 11, 2018

(54) DISPENSING DEVICE, KIT, AND METHOD FOR TISSUE AUGMENTATION

(71) Applicant: Ernesto Andrade, Bogota (CO)

(72) Inventor: Ernesto Andrade, Bogota (CO)

(21) Appl. No.: 15/480,299

(22) Filed: Apr. 5, 2017

(51) Int. Cl. A61F 2/12; A61M 5/34; A61M 5/32; A61M 5/00

(52) U.S. Cl. CPC A61F 2/12; A61M 5/002; A61M 5/3286; A61M 5/347

(57) ABSTRACT

A dispenser for tissue augmentation delivery. The dispenser includes a cannula that is prefilled with a bead composition for tissue augmentation. The bead composition is implanted into a patient's body. A plurality of beads are pumped out of the cannula via a delivery solvent, preferably in a single file configuration.

### BACKGROUND OF THE INVENTION

### Field of the Invention

The present invention is directed to dispensing device and a kit for a solution used in tissue augmentation that is used to administered a tissue augmentation solution into a patient's adipose tissue.

### Description of the Related Art

Over the past few decades, the field of cosmetic surgery has transitioned from what was once a relatively obscure practice, reserved primarily for severely wounded (military, accident victims, etc.) or wealthy consumers, into a more socially acceptable and financially affordable option that is now available to a majority of the population in economically developed countries.

The present day field of cosmetic surgery spans a wide variety of procedures and technologies, targeting different body parts and tissue types, ranging from hair and skin to breasts and buttocks. One particular area of cosmetic surgery commonly referred to as “augmentation” involves adding material (whether natural, synthetic, or a combination thereof) to a desired part of the body to achieve a desired size and/or shape. Breast or buttock enhancements are common examples of augmentation surgeries.

One primary method of accomplishing augmentation involves placement of an implant of predetermined size and shape into a desired treatment area. This is one of the most common augmentation methods, and although highly developed and largely proven, it still entails certain drawbacks. For instance, the precise size and shape of the implant must be determined prior to placement, which leaves little room for adjustment. From a practical standpoint, this limits the potential for fine tuning of the procedure and generally lacks the ability to provide for last-minute modifications to the patient's overall final appearance. Another problem associated with implants is that there is a distinct probability that the patient's body, over time, will ultimately develop an aversion to the implant such that the procedure will eventually need to be repeated with a replacement cosmetic implant. One of the most challenging aspects of repeating such a procedure is that it will likely be substantially invasive such that the patients are sometimes not physically fit for the surgery, or sometimes simply no longer desire to undergo the procedure.

### SUMMARY OF THE INVENTION

It is one object of the present invention to provide a pre-loaded dispensing cannula for use in tissue augmentation procedures that comprises a bead composition, wherein said bead composition comprises biocompatible semi-solid beads. In one preferred embodiment said semi-solid beads have a diameter of between 1 mm and 10 mm manufactured from a medical grade polymer material.

It is a further object of the present invention to provide a cannula that comprises an attachment end having an inlet port and an attachment assembly, a channel having an inner chamber; and a distal end having an outlet port, wherein said channel connects said inlet port and said outlet port, and wherein said channel has an inner chamber that holds said bead composition. In a further embodiment, the inner chamber has a diameter of about 1.0 mm to 10.0 mm, preferably 3.0 mm to 6.0 mm, more preferably 4.0 mm to 4.5 mm, and a capacity pf 2.5 mL. In some embodiments, the cannula 40 has an internal diameter of 4.2 mm. In another preferred embodiment, the channel has a length of about 8 to 16 inches, more preferably 10 to 14 inches, more preferably 12 inches.

Is another object of the present invention to provide a cannula wherein the biocompatible semi-solid beads are in a single line occupying a single space within the inner chamber diameter. In yet a further embodiment, the cannula comprises a delivery solvent, preferably saline solution.

Another object of the present invention is to provide a kit assembly that comprises a container housing a plurality of cannulas, wherein each of said cannulas comprise a bead composition, wherein said bead composition comprises biocompatible semi-solid beads. In one preferred embodiment, the container comprises a body, a top cap, a bottom cap, at least one cannula guide tray, preferably at least two cannula guide trays. In a preferred embodiment, the cannula guide tray comprises a plurality of apertures having a diameter slightly larger than an outer diameter of a channel of the cannula. In yet a further embodiment, the bottom cap is configured to prevent the biocompatible semi-solid beads from leaving the cannula. In another embodiment, the top cap comes in contact with an attachment assembly of the cannula and the bottom cap comes in contact with the distal end of the cannulas and prevents the biocompatible semi-solid beads from leaving the cannulas. In another embodiment, the kit assembly includes at least one syringe filled with delivery solvent and configured to be releasably attached to the cannulas.

It is another object of the present invention to provide a method of tissue augmentation, comprising a first step of preparing a tissue site for infiltration by injecting a tumescent fluid to the tissue. The method also provides a subsequent step of inserting a pre-loaded dispensing cannula, comprising a bead composition, wherein said bead composition comprises biocompatible semi-solid beads to the tissue site. In yet another preferred embodiment, the method includes a step of administering the biocompatible semi-solid beads to the tissue. In another embodiment, the method includes a step of making a small incision at the tissue site prior to inserting the preloaded cannula to the tissue site. In another embodiment, the method also includes a step of removing the cannula from the tissue site.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a schematic side view of one embodiment of the present invention.

FIG. 2A is a schematic side view of a cannula loaded with beads in accordance with one aspect of the present invention.

FIG. 2B is a schematic side view of a cannula without beads in accordance with one embodiment of the invention.

FIG. 3A is a schematic perspective view representation of one embodiment of a kit in accordance with the present invention.

FIG. 3B is a schematic plan view representation of another embodiment of a kit in accordance with the present invention.

FIG. 4 is a flow chart representation of a method of the present invention.

FIG. 5 is a schematic representation of a portion of the method of FIG. 4 utilizing the system of FIG. 1.

FIG. 6 is a graphical representation of the method of delivering a bead composition in accordance with one embodiment of the present invention.

Like reference numerals refer to like parts throughout the several views of the drawings.

### DETAILED DESCRIPTION OF THE PREFERRED EMBODIMENT

The invention summarized above may be better understood by referring to the following description, which should be read in conjunction with the accompanying claims and drawings in which like reference numbers are used for like parts. This description of an embodiment, set out below to enable one to build and use an implementation of the invention, is not intended to limit the invention, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent constructs and cell lines do not depart from the spirit and scope of the invention in its broadest form.

As shown in the accompanying Figures, the present invention is directed to a preloaded dispensing cannula 40 for use in tissue augmentation procedures. The preloaded dispensing cannula 40 contains a reliably safe bead composition 30 that is systematically administered into a patient's adipose (or “fat”) tissue. The bead composition 30 can be supplied in connection with dispensing apparatus 20, which is attached to the preloaded dispensing cannula 40 for dispensing the bead composition 30. The preloaded cannula 40 can be provided in the form of one or more kits 100. Each kit 100 can be customized for use in connection with a specific treatment or group of treatments.

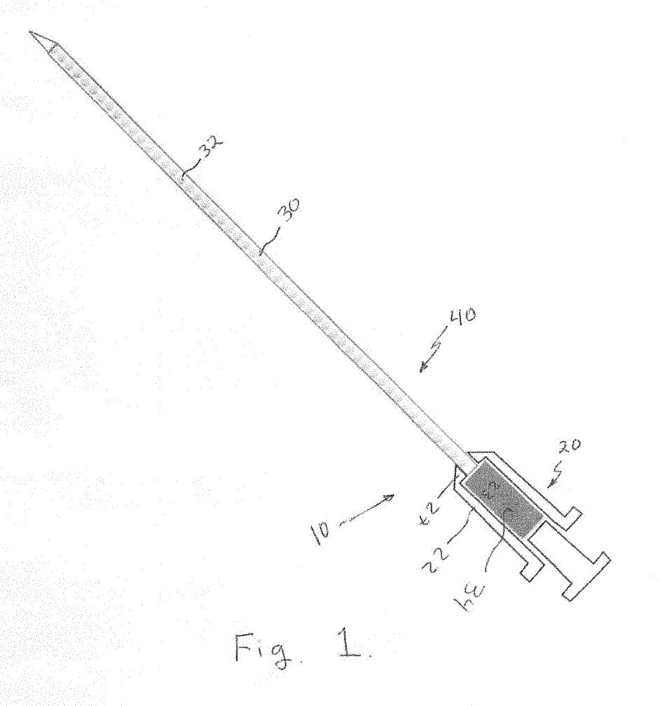

As seen in FIG. 1, the composition 30 is intended to be loaded into a dispensing cannula 40; preferably pre-packaged for use. The composition 30 primarily comprises a plurality of substantially spherical, biocompatible semi-solid beads 32. As used herein, the term “biocompatible” means generally safe for use as an implant material for use in the human body. As used herein, “semi-solid” means generally solid, in contrast to fluid, yet with a rubber-like consistency that generally mimics the feel and density of flesh. In a preferred embodiment, the biocompatible semi-solid beads 32 are manufactured from a medical grade polymer-based material of construction, such as medical grade silicone. However, other suitable materials with similar properties are also contemplated to be within the scope and intent of the present invention. Prior to use and/or loading into the dispensing cannula 40, the beads 32 are sterilized by a suitable method. By way of example, suitable sterilization methods can include, but are not limited to, chemical sterilization methods, Ethylene Oxide methods, irradiation methods, etc.

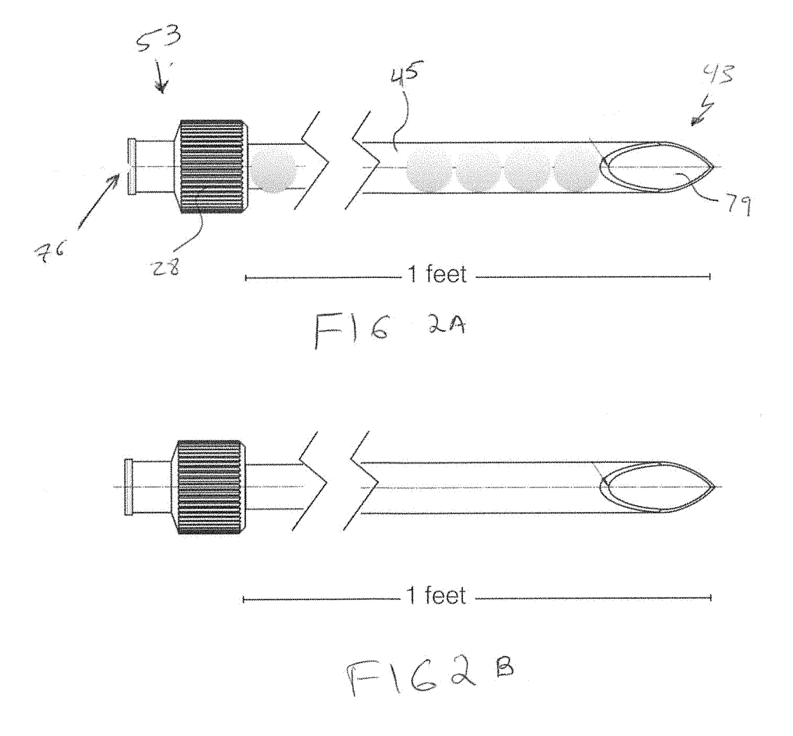

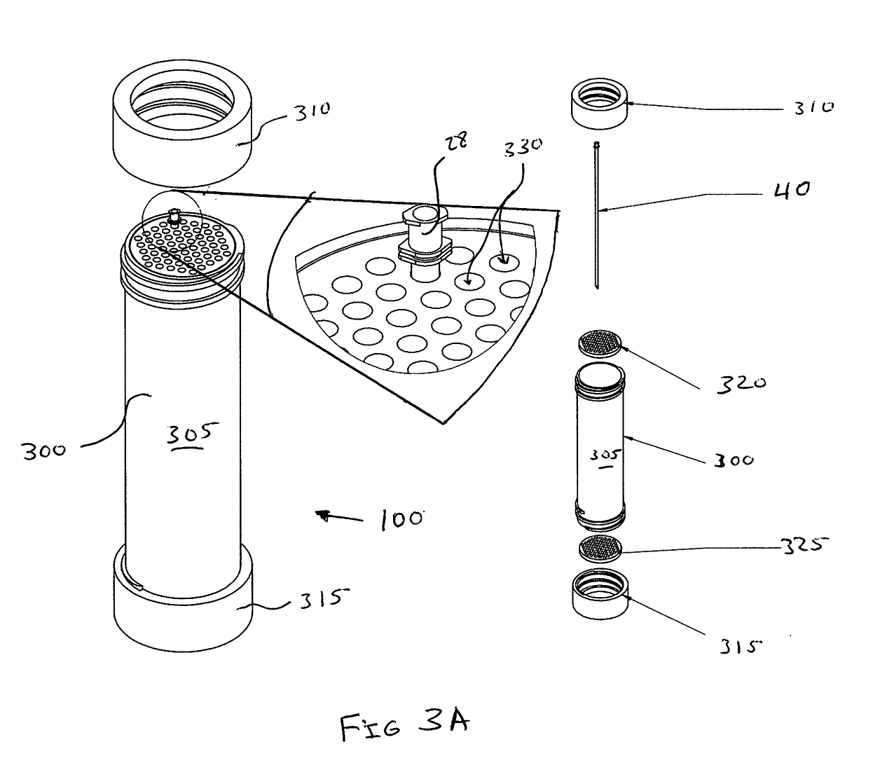

As used in this application the term “cannula”, as shown in FIG. 2, refers to a device that can be connected to a syringe for the delivery of the composition, such as a needle. In one preferred embodiment, the cannula 40 comprises an attachment end 53 having an inlet port 76 and an attachment assembly 28; a channel having an inner chamber 45; and a distal end 43 having an outlet port 79. The channel connects the inlet port 76 and the outlet port 79. The channel is configured to hold the bead composition 30. In one preferred embodiment, the cannula 40 has a length of about 8 to 16 inches, more preferably, 10 to 14 inches. In a further preferred embodiment the channel of the cannula 40 has a length of 12 inches. In a further embodiment, the inner chamber 45 has an internal diameter of about 1.0 mm to 10.0 mm, preferably about 3.0 mm to 6.0 mm, more preferably 4.0 mm to 4.5 mm, and a capacity pf 2.5 mL. In some embodiments, the cannula 40 has an internal diameter of 4.2 mm. The term “about,” as used herein when referring to a measurable value such as diameter, length, volume, and the like, is meant to encompass variations of 10, 1, 0.1, even 0.01 of the specified measurement.

It is emphasized that a primary inventive feature of the present invention involves the preloading of the beads 32, whose size range was previously thought too large to be practically administered given traditional delivery methods and the pressure differentials associated with the relevant target injection areas and to be held on the preloaded dispensing cannula 40. For instance, it had been generally acknowledged in the art that implant particles of a particular size range greater than just a fraction of a millimeter were simply too large to be injected for use in cosmetic augmentation of the type contemplated herein, as explained in application Ser. No. 14/968,303, which incorporated herein by reference in its entirety. However, the present inventive composition 30 enables the targeted infiltration of biocompatible semi-solid beads 32 each having an outer diameter in the range of about 1 millimeter to about 10 millimeters, utilizing a preloaded dispensing cannula 40. More specifically, the method 200, discussed in more detail below, involves infiltrating the beads 32 directly into a specific tissue, namely, the adipose tissue to achieve previously unattainable results. In contrast, previous methods generally focused on injecting implant material into inter-tissue layers, such as the areas between the muscle, skin, and adipose layers, respectively. Accordingly, the present invention utilizes a composition 30 having beads 32 in the aforementioned size range, wherein the more specific size of the beads 32 is selected to generally correspond to appropriate use in a desired target area.

Factors in choosing the appropriate bead size can involve size or volume of the target area as well as the desired effect of the infiltration. By way of example, one range of generally categorized bead size has an outer diameter in the range of about 1-2 millimeters; another in the range of about 3-4 millimeters. The present invention is primarily directed to a composition 30 having larger sized beads, and in particular, beads 32 in the range of at least 5 millimeters with ideally, all of the beads 32 to be used as tissue implants having a substantially uniform outer size. It is also within the spirit and scope of the present invention for the composition to include beads having larger sizes, such as an outer diameter of 6 millimeters, or more, whether 7 millimeters, 8 millimeters, 9 millimeters or 10 millimeters, with it again pointed out that effort made by others to date have not considered the use of such larger sized beads as a composition for tissue implants. The cannula 40 has an internal diameter that allows the beads to be loaded in a single line, i.e., the beads in the cannula 40 can occupy only a single space within the cannula's inner diameter 45.

Benefits of utilizing beads 32 of the aforementioned size ranges and cannulas that have an inner diameter 45 slightly larger than the beads' 32 diameter include resistance to, or avoidance of, shifting within the tissue; resistance to, or avoidance of, clumping; and resistance to, or avoidance of, absorption by the body. Furthermore, since the composition 30 directly infiltrates the fat tissue, the nature of the adipose tissue itself provides a smoothing effect which provides for greater aesthetic tolerances despite the non-traditional size of the beads 32.

In at least one embodiment, the composition 30 also includes a delivery solvent 34 structured to provide sanitary lubrication of the beads 32 relative to one another and relative to an interior portion of the dispensing cannula 40. The delivery solvent 34 can also serve to lubricate the beads 32 upon infiltration into the soft tissue. The plurality of beads 32 is immersed in the delivery solvent 34 as the composition 30 is contained within an inner chamber 45 of a dispensing cannula 40. Preferably, the delivery solvent 34 comprises primarily saline solution, typically 0.9 percent Sodium Chloride (0.9% NaCl) dissolved in water, although a wide variety of other possible concentrations are contemplated to be implemented, as appropriate. It is also within the scope and intent of the present invention to utilize delivery solvents 34 that comprise other suitable chemical compositions.

With continued reference to the system 10 depicted in FIG. 1, the dispensing apparatus 20 can comprise a syringe configuration as shown. In this embodiment, the syringe 20 comprises a body 22 which encloses an inner chamber 23 structured to releasably contain the delivery solvent 34 or other appropriate solvent. The syringe 20 includes a plunger 24 having an outer end portion 25 that a user presses inward to control dispensing of the composition 30, and an oppositely disposed inner end portion 26 situated on the interior of the chamber 23 which serves to force the delivery solvent 34 out of the chamber 23 via the neck 27, pushing the composition 30 out of the dispensing cannula 40. The overall shape of the syringe 20 is typical of syringes commonly available in the art. The size of the syringe 20 will depend on the desired volume of delivery solvent 34 for a given application. The syringe 20 is manufactured from a variety of suitable materials readily available to those knowledgeable in the art. By way of example only, one such material can include PETE 1, a medical grade thermoplastic polymer resin often used to construct syringes and other medical apparatus.

The dispensing apparatus 20 further comprises the dispensing cannula 40 structured to precisely deliver the plurality of beads 32 to the desired location. As seen in FIG. 2, the cannula 40 comprises an inner diameter 45. In at least one embodiment, the inner diameter 45 of the cannula 40 is selected to accommodate passage of a stream of individual beads 32 having a corresponding bead size. The cannula 40 also preferably comprises a suitable attachment assembly 28 that can facilitate side by side packaging. The cannula 40 can thus be connected to the syringe body 22 by connecting the attachment assembly 28 to the corresponding neck portion 27 of the syringe 20. In at least one embodiment, the attachment is achieved by mechanical threading, although a variety of other suitable interconnection means are contemplated as well. FIG. 2 further depicts a rounded tip 43 which serves to facilitate insertion into the desired target area of adipose tissue. The cannula 40 can comprise any suitable material of construction readily available in the art, including, but not limited to, metals, plastics, composites, etc.

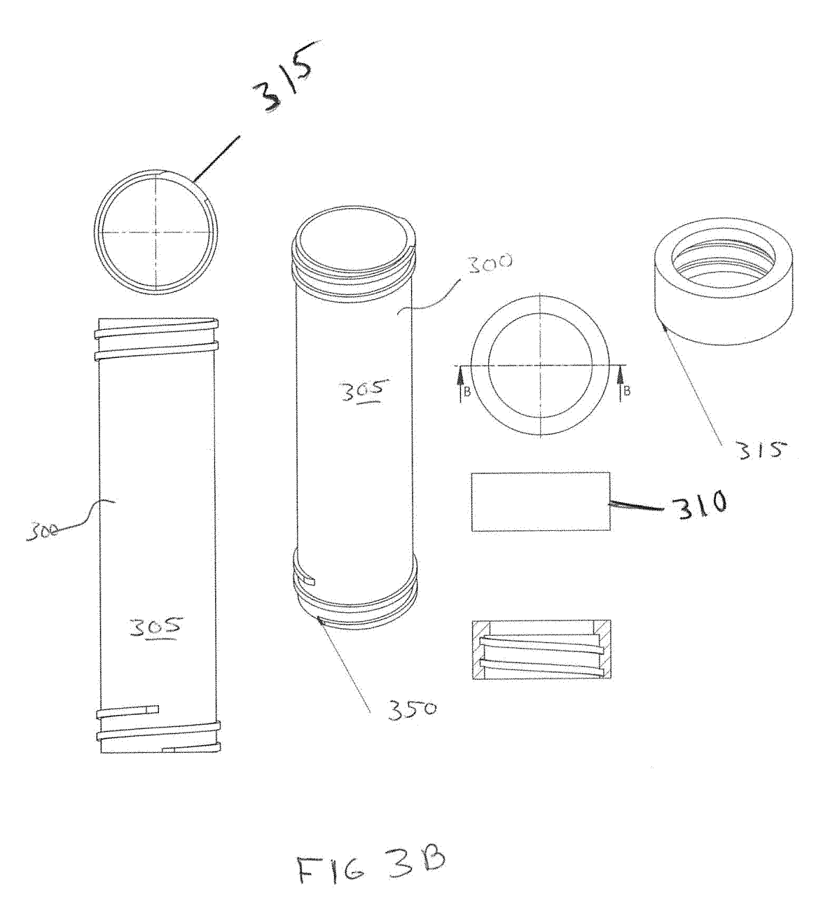

With primary reference to the generic embodiments of FIGS. 3A and 3B, the various components of the kit assembly 100. The kit assembly comprises a container housing a plurality of cannulas, wherein each of said cannulas comprise a bead composition, and wherein said bead composition comprises biocompatible semi-solid beads. In one preferred embodiment, the kit assembly comprises a container 300. In the preferred embodiment shown, the container 300 is cylindrical. A person of ordinary skill in the art would recognize that various other shapes can be utilized without departing from the spirit of the present invention. The container 300 has a body 305, a top cap 310, and a bottom cap 315. The container further includes at least one, preferably two, cannula guide trays 320 and 325 configured to accept a plurality of cannulas 40. The top cannula tray 320 is configured to be placed in the container 300 near the top cap 310, while the bottom tray 325 is configured to be placed near the bottom cap 315.

The top cannula tray 320 and the bottom cannula tray 325 comprise a plurality of apertures 330 that have a diameter slightly larger than the outer diameter of the channel of the cannulas 40. The attachment assembly 28 (or louver lock) prevents the cannulas 40 from going through the apertures 330 of the trays 315 and 320. The top cannula tray 320 and the bottom cannula tray 325 are aligned to allow the cannulas 40 to be organized in the container 300. In a preferred embodiment, the top cannula tray 320 and the bottom cannula tray 325 have the same diameter of the inner wall of the container 300 and are both glued to the inside wall of the container once the apertures 330 of both trays are aligned. It is contemplated that, in some embodiments, an adhesive is used to secure the trays 315 and 320 to the walls of the container 300. In other preferred embodiments, the trays 315 and 320 are secured to the walls of the container 300 by the use of heat that melds the elements together. The container 300 has a length equal or slightly larger than the cannulas 40. In one preferred embodiment the cannula 40 has a length of 20 cm and the container 300 has the same length between the top cannula tray 320 and bottom cannula tray 325. Once all the cannulas 40 are loaded onto the container 300, each cannula 40 is loaded with the appropriate number of beads 32. The bottom cap 315 prevents the beads 32 from departing the cannula 40. After the cannulas 40 are loaded, the container is filled with the delivery solvent 34, such as saline solution. Each component of the kit can be separately sterilized using standard sterilization techniques. Once assembled, the completed kit can be further sterilized using standard sterilization techniques.

The top cap 310 comes in contact with the attachment assembly 28 and preventing the cannulas from moving once the top cap is secured. The bottom cap 315 is configured to contact the distal end 43 of the cannulas 40 and prevent the beads 32 from leaving the cannulas 40 once loaded. In order to prepare the kit 100, the following procedure is used. The bottom cannula tray 325 and the bottom cap 315 are secured to the body 305 of the container 300. The top cannula tray 320 is then placed on the upper end of the body 305 of the container 300. A plurality of cannulas 40 are loaded in the container 300 by inserting the cannulas 40 through the apertures 330 of the top cannula tray 320 and bottom cannula tray 325. In one preferred embodiment, a single container can accommodate between 50 to 80 cannulas 40, preferably between 55 and 65 cannulas 40, more preferably 61 cannulas 40.

In some embodiments, the pre-loaded cannulas 40 are provided outside of the container 300 and may include separate packaging. By way of example, in one embodiment, the kit 100 can comprise one 2.5 cc cannula 40 pre-filled with a composition 30 that includes a plurality of 2 millimeter diameter medical grade silicone spheres 32 immersed in a delivery solvent 34. The respective parameters of such a kit may correspond to a specific desired type of treatment such as, in this example, a smaller type of body area which may include the chin and/or neck area. In other cases, a kit 100 for augmentation of a larger body area, such as the gluteal area, can include a plurality of larger pre-loaded cannulas 40. One specific example includes ten 20.0 milliliter syringes 20, each pre-filled with a delivery solvent 34, and pre-filled cannulas 40 filled with 3 or 4 millimeter diameter semi-solid spheres 32 immersed in delivery solvent 34. Generally, it is preferable from a delivery standpoint that the selected beads 32 for a given volume of composition 30 each comprise a substantially uniform sized outer diameter.

The kits 100, like the composition 30, are assembled in a sterile environment and comprise safe, sterile materials of construction suitable for medical applications. In particular, the container 300 can comprise a variety of shapes, configurations, and materials. As seen in FIGS. 3A-3B, the containers 300 typically comprise a solid, generally cylindrical configuration and are ordinarily made of a polyethylene material of construction, such as PETE 1, or similar type of plastic material. The corresponding caps 310 and 315, as well as the optional separate packaging, can also be made of plastic or other suitable material, such as polyethylene spun fiber or TYVEK sheets. Further, the tops 310 and 315 can attach to the container 300 in a variety of ways, such as screw configuration as shown, but other attachment configurations as understood by a person of ordinary skill can be utilized such as built-in adhesives, snap-on fit configuration, separate attachment means, etc. In one preferred embodiment, the kit 100 includes 40 pre-filled needles for a total volume of 100 ml of solution 30.

The various components of the kits 100 can be sterilized by variety of methods including, but not limited to, chemical sterilization methods, Ethylene Oxide methods, irradiation methods, etc. In at least one preferred embodiment, the assembled kits 100 are sterilized utilizing a STERRAD radiation sterilization system.

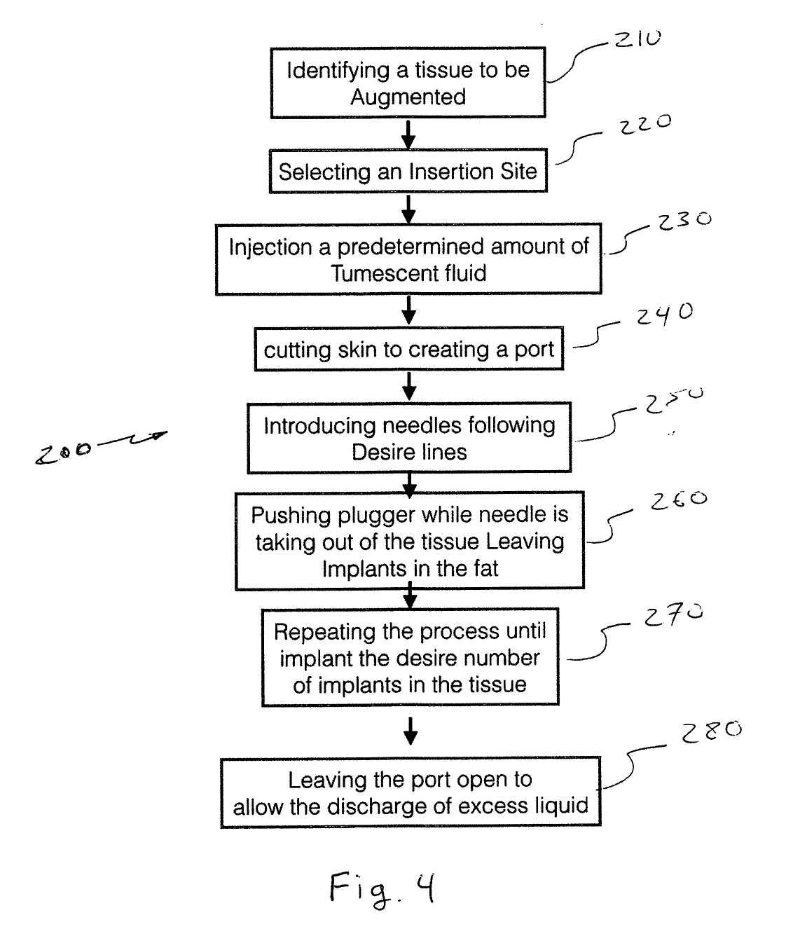

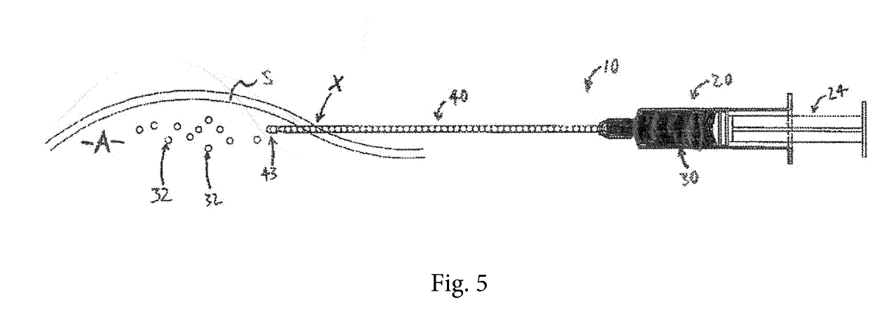

With primary reference now to FIGS. 4, 5, and 6, the present invention contemplates associated methods 200, utilizing the above-described cannulas 40 and/or kits 100. Turning first to FIG. 4, the method 200 associated with the present invention begins at 210, wherein a surgeon or other practitioner identifies the local tissue to be augmented (e.g., adipose tissue of the chin, cheek, breast, buttocks, etc.). As seen in FIG. 5, the adipose tissue “A” generally lies beneath the skin tissue “S.”

As illustrated at 210, the method 200 further comprises selecting an insertion site “X” and appropriately marking it for entry of the cannula 40. Directional indication markers, such as those indicating one or more directional axes, may also be utilized to facilitate the administration of the composition 30.

Next, a predetermined amount of tumescent fluid or similar type of anesthetic fluid, such as Klein Solution, is injected via traditional means into the target area to prepare the tissue for infiltration of the composition 30, as shown at 230. The tumescent fluid acts as a local anesthetic and also tends to firm up the target tissue. The tumescent fluid can comprise a predetermined amount of anesthetic (such as Xylocaine, e.g. 0.1% solution) as well as predetermined amounts of other optional substances, such as vasoconstrictor (e.g. Epinephrine), Sodium Bicarbonate, etc. The procedure 200 may also be performed by administering other types of general, regional, or local anesthesia to the patient. Ordinarily, a predetermined amount of time (e.g., 10 minutes, 15 minutes, etc.) will be allowed to pass subsequent to injecting the tumescent fluid before infiltrating the tissue with the augmentation composition 30. This provides sufficient time for the anesthetic to take effect.

Once the anesthetic takes effect, a small incision is made into the skin S at the insertion site X, as shown at 240. The incision permits the passage of the cannula 40 into the adipose tissue A, for infiltrating the tissue A with the augmenting composition 30, which is generally indicated at 250. By way of example, the incision can be made with a #15 scalpel or similar-type surgical tool to provide sufficient clearance for inserting an appropriate portion of the pre-loaded cannula 40 while minimizing the subsequent effort needed to close the incision upon completion of the procedure. In some embodiments, the cannula 40 has a slanted tip that allows it to be inserted into the skin S without the need of an initial incision.

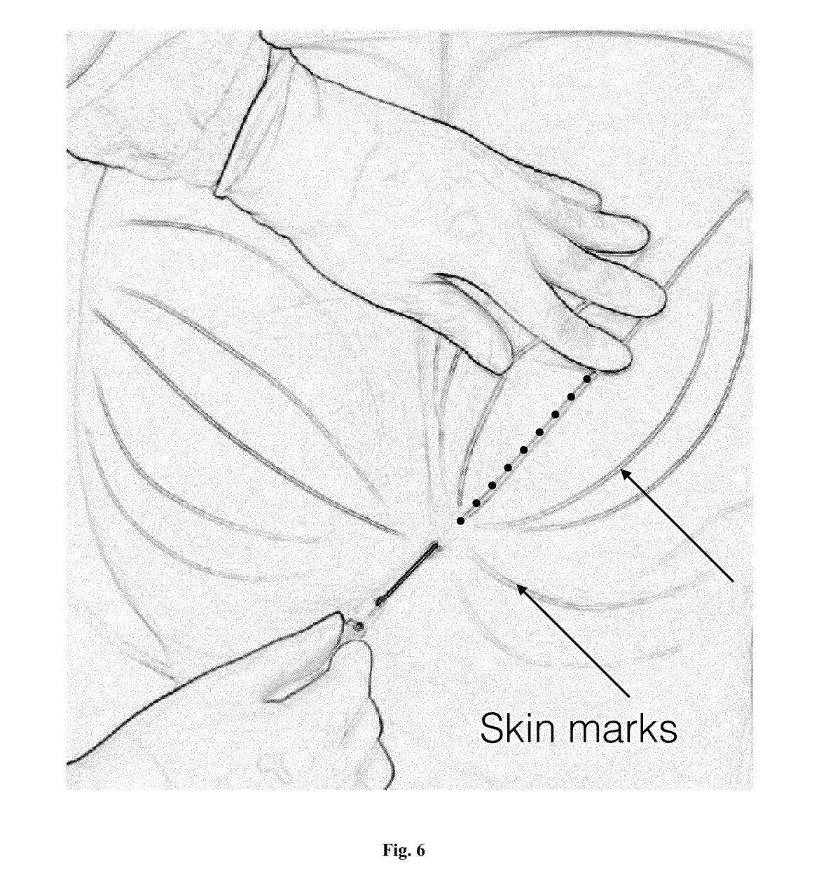

Next, as also seen in FIGS. 5 and 6, the distal end 43 of the cannula 40 is positioned to a maximum desired depth within the target area of the tissue A. The composition 30 is then administered into the adipose tissue A, as generally indicated at 260, by pushing the plunger as the cannula 40 (or needle) is removed from the insertion site leaving the implants in the desired location. As also shown in FIG. 5, the depositing of the composition 30 into the tissue A is preferably achieved by controlled dispensation as shown on FIG. 4, wherein the distal end 43 of the cannula 40 is simultaneously withdrawn away from the interior distal location of insertion and back towards the insertion site X as the composition 30 is dispensed. In at least one embodiment, the dispensation of the composition 30 is controlled by depressing the plunger 24 portion of a syringe 20 slowly and evenly at an appropriate rate while the distal end 43 of the cannula 40 is slowly withdrawn towards the insertion site X, up to within approximately 1-2 inches thereof. This technique can be repeated until a desired quantity of composition 30 is infiltrated into the tissue A, as provided in 270. In this way, the composition 30 is distributed to achieve the desired effect.

While in some applications a single, pre-loaded cannula 40 will contain a sufficient amount of composition 30 to achieve the desired degree of augmentation, other cases may require use of a plurality of pre-loaded cannulas 40, as at 220, perhaps of varied volumes and/or containing varied amounts of the augmentation composition 30, as described above. In cases where a kit 100 is utilized, if the procedure 200 is completed without using all of a plurality of cannulas 40, then the container 300 can be sealed using the appropriate top cap 310 for later use. The kit 100 can also be re-sterilized accordingly.

Once the desired result is achieved, the insertion site X is left open to allow the discharge of excess fluid as provided at 280. In some preferred embodiments, the puncture through which the needle is inserted does not require any closure as it will close naturally. In other embodiments, the puncture may be closed by appropriate medical procedures, such as with an appropriate suture or other surgical closure means. For instance, as one example, a Polypropylene suture, (e.g., PROLENE 5-0 suture) can be used. Following closure of the insertion site X, the patient should be kept at rest in an appropriate decubitus position for a predetermined amount of time, to allow for initial recovery from the rigors of the procedure 200.

Most existing methods for tissue augmentation utilize solid or semi-solid implants, such as medical grade silicone. For example, a current method for gluteal tissue augmentation requires the implantation of a single implant, sometimes filled with implantable material, at the site of implantation. In one example, in order to obtain an increase of 300 cubic centimeters of gluteal enhancement, a 300 cubic centimeter implant is applied through an incision in the gluteal region leaving a significant scar. According with one embodiment of the present invention, on the other hand, the same volume of augmentation is accomplished through the delivery of 9,000 single implants.

A particular advantage of the present method is that the individual implants are delivered through a small aperture created by the cannula, which closes on its own leaving little or no scaring. Furthermore, unlike traditional implantation methods, the implants of the present invention are delivered within a specific tissue as opposed to being delivered between various layers of tissue. A traditional breast implant is placed between the mammary gland and the pectoral muscle. In one embodiment of the present invention, the implants are delivered within the mammary gland tissue or within the pectoral muscle tissue. More specifically, the implants of the present invention can be delivered to the within the fatty tissue of the breast or other target area.

Existing methods in which utilize either 1) peristaltic pumps, 2) transferred using a syringe and a needle, or 3) preloaded loaded syringes. The present invention, on the other hand, utilizes preloaded cannulas that can connected to syringes that do not have implantable material but a transfer medium such as saline solutions. No other methods utilize preloaded cannulas for tissue augmentation.

It should also be noted that although the present invention is primarily intended for use on humans, it could be applied to other beings, such as to dogs, horses, etc., should such a need arise. Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A pre-loaded dispensing cannula for use in tissue augmentation procedures, comprising:
a bead composition, wherein said bead composition comprises biocompatible semi-solid beads.

2. The cannula of claim 1, wherein said semi-solid beads have a diameter of between 1 mm and 10 mm.

3. The cannula of claim 1, wherein said semi-solid beads are manufactured from a medical grade polymer material.

4. The cannula of claim 1, comprising
an attachment end having an inlet port and an attachment assembly,
a channel having an inner chamber; and
a distal end having an outlet port, wherein said channel connects said inlet port and said outlet port, and wherein said channel has an inner chamber that holds said bead composition.

5. The cannula of claim 4, wherein said inner chamber has a diameter of about 1.0 mm to 10.0 mm, preferably 3.0 mm to 6.0 mm, more preferably 4.0 mm to 4.5 mm, preferably 4.2 mm and a capacity pf 2.5 mL.

6. The cannula of claim 5, wherein said channel has a length of about 8 to 16 inches, more preferably 10 to 14 inches, more preferably 12 inches.

7. The cannula of claim 1, wherein the biocompatible semi-solid beads are in a single line occupying a single space within the inner chamber diameter.

8. The cannula of claim 1, further comprising a delivery solvent.

9. The cannula of claim 8, wherein the delivery solvent is a saline solution.

10. A kit assembly, comprising:
a container housing a plurality of cannulas, wherein each of said cannulas comprise a bead composition, wherein said bead composition comprises biocompatible semi-solid beads.

11. The kit assembly of claim 10, wherein said container comprises a body, a top cap, a bottom cap, at least one cannula guide tray.

12. The kit assembly of claim 11, comprising at least two cannula guide trays.

13. The kit assembly of claim 11, wherein the cannula guide tray comprises a plurality of apertures having a diameter slightly larger than an outer diameter of a channel of the cannula.

14. The kit assembly of claim 11, wherein the bottom cap is configured to prevent the biocompatible semi-solid beads from leaving the cannula.

15. The kit assembly of claim 11, wherein the top cap comes in contact with an attachment assembly of the cannula and the bottom cap comes in contact with the distal end of the cannulas and prevents the biocompatible semi-solid beads from leaving the cannulas.

16. The kit assembly of claim 10, further comprising at least one syringe filled with delivery solvent and configured to be releasably attached to the cannulas.

17. The kit assembly of claim 11, wherein the container has a cylindrical shape.

18. A method of tissue augmentation, comprising:
preparing a tissue site for infiltration by injecting a tumescent fluid to the tissue;
inserting a pre-loaded dispensing cannula, comprising a bead composition, wherein said bead composition comprises biocompatible semi-solid beads to the tissue site;
administering the biocompatible semi-solid beads to the tissue.

19. The method of claim 18, further comprising making a small incision at the tissue site prior to inserting the preloaded cannula to the tissue site.

20. The method of claim 18, further comprising removing the cannula from the tissue site.
